(19) United States
(12) Patent Application Publication
(10) Pub. No.: US 2010/0081968 A1
(43) Pub. Date: Apr. 1, 2010

(54) Test Strip With Integrated Lancet

(75) Inventors: Gary T. Neel, Weston, FL (US); Allan Javier Caban, Boynton Beach, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Home Diagnostics, Inc.

(21) Appl. No.: 12/567,201

(22) Filed: Sep. 25, 2009

Related U.S. Application Data
(63) Continuation-in-part of application No. 11/711,621, filed on Feb. 28, 2007.
Application No. 11/711,621 is a continuation-in-part of application No. 11/181,778, filed on Jul. 15, 2005.

(51) Int. Cl. A61B 5/151

(52) U.S. Cl. 600/583

(57) ABSTRACT

The present disclosure provides methods and systems to collect blood from a patient using a lancet integrated with a test strip. The present disclosure provides integrated test strips having an integrated lancet, as well as meters for actuating the lancet integrated within a test strip. The analyte test strip comprises a first test strip substrate material and a second test strip substrate material. At least one test strip substrate material comprises an elongate cavity. The test strip further comprises a reaction test site for testing analyte concentrations in blood. The test strip further comprises at least one opening communicating with the at least one cavity. The test strip and integrated lancet can be sterilized and then sealed in a package.

### CROSS-REFERENCE TO RELATED APPLICATIONS

This application is a continuation-in-part of co-pending U.S. application Ser. No. 11/711,621, filed Feb. 28, 2007, and a continuation-in-part of co-pending U.S. application Ser. No. 11/181,778, filed Jul. 15, 2005. The content of both applications is incorporated herein by reference in its entirety.

### TECHNICAL FIELD

The present disclosure pertains to the field of diagnostic testing and, more particularly, to diagnostic testing systems using electronic analyte meters.

### BACKGROUND

Electronic testing systems are commonly used to measure or identify one or more analytes in a sample. Such testing systems can be used to evaluate medical samples for diagnostic purposes and to test various non-medical samples. For example, medical diagnostic meters can provide information regarding the presence, amount, or concentration of various analytes in human or animal body fluids. In addition, diagnostic test meters can be used to monitor analytes or chemical parameters in non-medical samples such as water, soil, sewage, sand, air, or any other suitable sample.

Diagnostic testing systems typically include both a test medium, such as a diagnostic test strip, and a test meter configured for use with the test medium. Suitable test media may include a combination of electrical, chemical, and/or optical components configured to provide a response indicative of the presence or concentration of an analyte to be measured. For example, some glucose test strips include electrochemical components, such as glucose specific enzymes, buffers, and one or more electrodes. The glucose specific enzymes may cause a reaction between glucose in a sample and various chemicals on a test medium, thereby producing an electrical signal that can be measured with the one or more electrodes. The test meter can then convert the electrical signal into a glucose test result. Such enzymes may include glucose dehydrogenase, glucose oxidase, etc.

Diagnostic testing systems have improved significantly in recent years. For example, test meters have become smaller and faster, and the amount of blood or other fluid needed to obtain accurate test results has decreased. However, although these improvements have made testing more convenient for patients, current systems have some drawbacks. For example, current systems and devices for monitoring blood glucose levels in diabetic patients require patients to carry at least three devices: a lancet, a blood glucose meter, and test strips; and the need to carry three separate items can be inconvenient and cumbersome. In addition, carrying more components makes it easier to misplace or lose a component. Further, systems that employ separate lancets often include lancets that can be reused. However, reusing the same lancet is less sanitary than using a new, disposable lancet each time. In addition, repeated use of the same lancet can cause the lancet to become dull over time and cause more pain to the patient upon use.

While current methods and systems facilitate the self monitoring of analyte concentrations in blood or a bodily fluid, there is a need for additional features and improvements, including systems with fewer components and more efficient/advanced methods of manufacturing. The present invention is directed at overcoming one or more shortcomings of the prior art of meters, lancets, and test strips.

### SUMMARY

The present disclosure provides methods and systems to collect blood from a patient using a lancet integrated with a test strip. In traditional systems, the various components of a blood collection system (i.e., meter, test strip, and lancet) are separate objects. However, the present disclosure provides integrated test strips having an integrated lancet, as well as meters for actuating the lancet integrated within a test strip.

In one aspect of the present disclosure, an analyte test strip system may be composed of a test strip including at least one test strip substrate and an elongate cavity extending through at least one of the substrates. The cavity may be adapted to receive a lancet and the test strip can have an opening through which the lancet may be deployed. The test strip can also comprise a reaction site where the patient's blood can be collected. The test strip may be sealed in a package.

Another aspect of the present disclosure includes producing an analyte test strip system that integrates a test strip with a lancet. The method can include selecting a first test strip substrate material followed by selecting a second test strip substrate material. At least one elongate cavity can be formed in at least one of the first or second test strip substrate materials. A lancet material may be selected and cut into an elongate shape in order to fit into the elongate cavity formed in the at least one substrate material. The lancet may be disposed into the elongate cavity, and the first and second substrate materials may be mated to one another. The test strip with the integrated lancet may be sterilized and then sealed in a package.

### BRIEF DESCRIPTION OF THE DRAWINGS

The accompanying drawings, which are incorporated in and constitute a part of this specification, provide exemplary embodiments of the invention and, together with the description, serve to explain the principles of the invention.

FIG. 1 illustrates a perspective view of an analyte meter, according to an exemplary disclosed embodiment.

FIG. 2A illustrates a bottom view of a test strip, according to an exemplary disclosed embodiment.

FIG. 2B illustrates a perspective view of the test strip of FIG. 2A, according to an exemplary disclosed embodiment.

FIG. 2C illustrates a side view of the test strip of FIG. 2A, according to an exemplary disclosed embodiment.

FIG. 3 illustrates a perspective view of a test meter including a lancet actuation mechanism, according to an exemplary disclosed embodiment.

FIG. 4 illustrates a flow chart of a process for producing an analyte test strip system including a test strip with an integrated lancet, according to an exemplary disclosed embodiment.

FIG. 5 illustrates a perspective view of a test strip partially disposed in a ouch, according to an exemplary disclosed embodiment.

### DETAILED DESCRIPTION OF THE DRAWINGS

References will now be made in detail to the exemplary embodiments of the invention, examples of which are illustrated in the accompanying drawings. Wherever possible, the same reference numbers will be used throughout the drawings to refer to the same or like parts.

The present disclosure provides a test strip having an integrated lancet. This test strip with integrated lancet can be used to collect a sample of blood from a patient and then, in conjunction with an analyte meter, to measure the analyte content of the collected blood. In some embodiments, the test strip with integrated lancet can be mated with an analyte meter configured to actuate the lancet in order to pierce a patient's skin to collect a blood sample. Further, in some embodiments, the blood may be collected on the same test strip while still mated with the analyte meter in order to detect or measure an analyte in the blood.

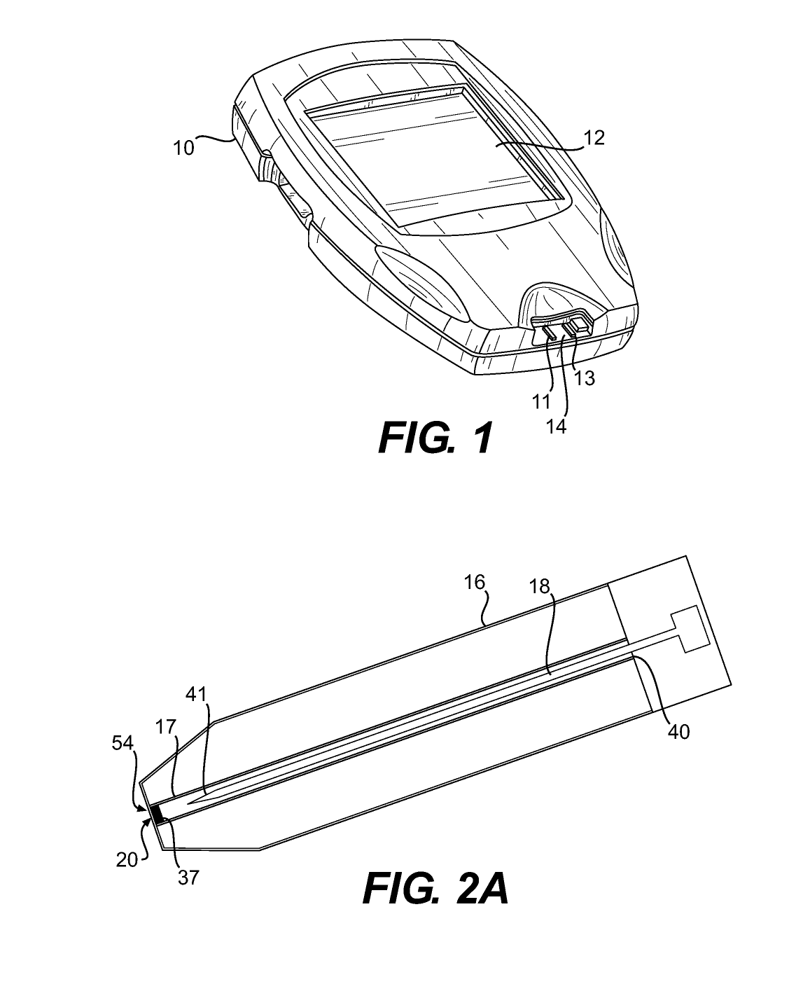

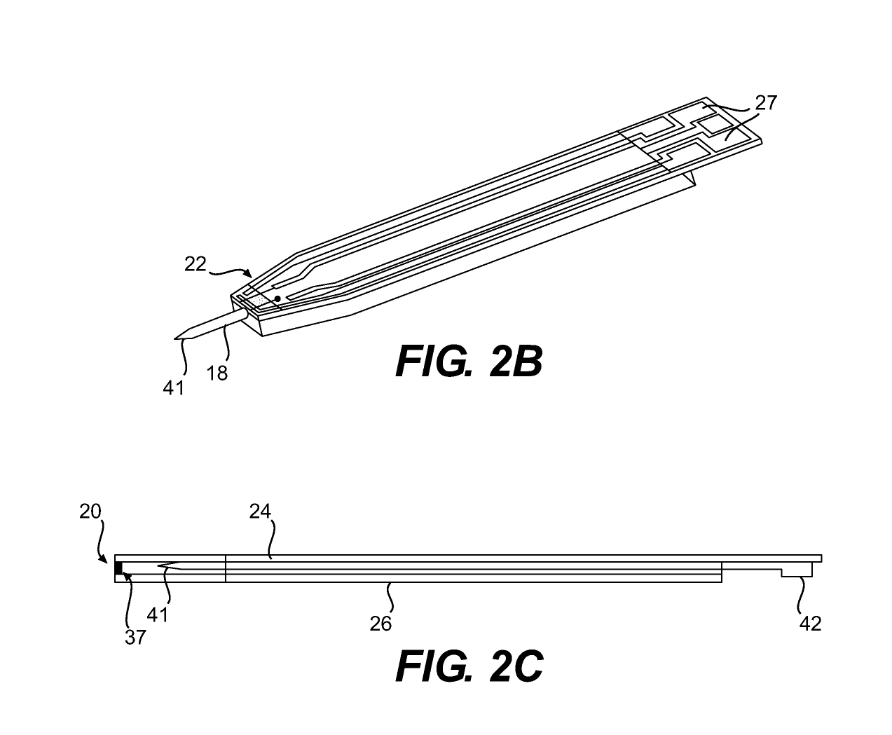

FIG. 1 illustrates a perspective view of an analyte meter 10, according to an exemplary disclosed embodiment. The analyte meter 10 can be configured to measure or detect blood analytes and to display relevant information to a user. As shown in FIG. 1, the analyte meter 10 comprises several components, including a display system 12 and a test strip interface 14. Display system 12 can be configured to communicate test results to a user using visual and/or audio systems. Further, the test strip interface 14 may be configured to mate with a test strip 16 (shown in FIGS. 2A-2C) to secure the test strip to the meter 10. Further, the interface 14 can include one or more electrodes 11, 13 configured to form an electrical connection with a mated test strip.

The analyte meter 10 can be used to detect or measure the concentration of one or more analytes. The one or more analytes may include a variety of different substances, which may be found in biological samples, such as blood, urine, tear drops, semen, feces, gastric fluid, sweat, cerebrospinal fluid, saliva, vaginal fluids (including suspected amniotic fluid), culture media, and/or any other biologic sample. In some embodiments the biologic sample can include blood and the analyte can include glucose.

As noted, FIG. 1 also illustrates the test strip interface 14 having electrical meter components with one or more electrodes 11, 13. The test strip interface 14 can be configured to mate with the test strip 10, and the one or more electrodes 11 13 on the test strip interface 14 may be configured to engage electrical components of a test strip, thereby facilitating measurement of electrical signals, i.e., currents or voltages, which may be produced by an electrochemical reaction within a test strip reaction test site and may represent the concentration of an analyte within a collected sample. The one or more electrodes 11, 13 can include a number of electrode types and configurations. For example, the one or more electrodes 11, 13 can include a cathode 11 and an anode 13, as well as one or more additional electrodes. The specific number and type of electrodes may be selected based on the desired testing modality, the specific analyte meter type being used, and any other suitable parameter. Further, the one or more electrodes 11, 13 can include a number of shapes and configurations. For example, the one or more electrodes 11, 13 can include planar electrodes, interdigitated electrodes, or any other suitable electrode pattern. Further, it will be understood that although the disclosed integrated test strip 16 and lancet 18 are described for use with an electrical test meter 10, any suitable testing system may be used. For example, suitable meters can include electrochemical systems, optical systems, electrochemiluminescent systems, radioactive assays, and/or any other suitable system in which an integrated test strip may be desirable.

FIG. 2A illustrates a bottom view of a test strip 16, according to an exemplary disclosed embodiment. As shown, the test strip 16 includes an elongate cavity 17 configured to hold a lancet 18. The test strip 16 also includes an opening 20 on one end 54 through which a sharp end 41 of the lancet may be advanced and retracted to pierce the skin of a patient proximate the opening 20. The test strip 16 may be configured to mate with a meter interface 14 at a second end 43 of the test strip 16. The second end 43 may further include one or more electrodes 27 (as shown in FIG. 2B) that can form an electrical connection with the meter electrodes 11, 13.

The test strip 16 may comprise a mechanism for adjusting the depth of penetration of the lancet 18. This may prevent the lancet 18 from piercing a patient's skin too deeply, and allow deeper penetration for patient's with thicker skin or less blood circulation. This mechanism includes a knob configured to adjust to an arm to physically stop the lancet from protruding past a certain distance. The mechanism can alternatively include a stopper adjustable by a sliding mechanism.

FIGS. 2B and 2C show the test strip 16 with integrated lancet 18 mated to an actuation mechanism 21 in the analyte meter 10. As noted previously, the lancet 18 can be advanced through the opening 20 at the distal end 54 of the test strip 16 in order to pierce the skin of a patient proximate the opening 20. For example, FIG. 2B shows the sharp, pointed end 41 of the lancet 18 pushed out of the test strip 16 by the actuation mechanism 21. Further, as shown in FIG. 2C, the lancet 18 may be retracted into the test strip 16 after piercing a patient's skin, thereby preventing further exposure of the sharp point 41 to a patient.

The lancet 18 in the test strip 16 can be made of a biocompatible plastic or a biocompatible metal. The biocompatible plastic can include a number of suitable types of polymeric materials including, but not limited to, thermosets, elastomers, or other polymeric materials. Further, suitable biocompatible metals can include, for example, stainless steel, titanium, etc. In addition, the lancet 18 can also be formed from various composite materials.

The lancet 18 may be manufactured using a number of suitable production processes. For example, the lancet can be fabricated using known metal processing techniques, such as casting or forging, or for the case of polymeric materials, any suitable polymer processing system can be used, including, for example, injection molding.

As noted previously, the lancet 18 can have a sharp, pointed end 41 that can be used to pierce a patient's skin in order to collect blood. The test strip end 43 that will be engaged with the analyte meter can include a hole 40 for the lancet's mating end 42. The lancet's mating end 42 will be configured to engage the actuation mechanism 21 in the analyte meter 10.

In some embodiments, the test strip 16 can include a membrane 37 that covers and fluidly seals the opening 20. The membrane 37 may help secure the lancet 18 in the cavity 17 until the lancet 18 is needed for use. Further, the membrane 37 can prevent the lancet from becoming contaminated during storage. The membrane 37 can include a variety of suitable materials. For example, the membrane 37 can include any suitable polymer or composite material that can be pierced by the lancet 18 when the lancet 18 is actuated by the actuation mechanism 21. The covering 37 may be impermeable or semipermeable to gas or liquid. For example, suitable materials include polymer thin films, polyethylene, latex, etc.

FIG. 2B illustrates a perspective view of the test strip of FIG. 2A, according to an exemplary disclosed embodiment. As shown, the strip 16 can include a reaction test site 22 configured to receive a blood sample collected after piercing a patient's skin with the lancet 18. The test site 22 can include one or more substances configured to react with one or more analytes. For example, the reaction test site 22 may include one or more enzymes configured to react with an analyte such as glucose. Furthermore, the reaction test site 22 may include other additives, including salts, buffers, enzyme stabilizers, electrochemical mediators, color indicators, and/or any other chemical needed to facilitate production of a suitable test reaction.

The reaction test site 22 may have a shape and size configured to hold certain substances needed to react with an analyte to be tested. For example, the reaction test site 22 may include a well configured to secure a certain sample volume. In addition, the reaction test site 22 may include various configurations that can facilitate sample acquisition, proper sample placement, or needed fluid flow.

In addition, the test strip 16 can include one or more electrodes 27. These electrodes 27 can be configured to engage corresponding electrodes on an analyte test meter to form an electrical connection with the test meter 10, thereby allowing a reaction that occurs at the test site 22 to be correlated with a blood analyte concentration.

It should be noted that although the test strip 16 is shown as a rectangularly shaped strip, the test strip 16 can include a variety of suitable shapes and sizes as long as the test strip 16 can include an elongate cavity 17 and lancet 18. For example, the test strip 16 can be in the form of ribbons, tabs, discs, or any other suitable form. Further, as noted above, the test strip 16 can also be configured for use with a variety of suitable testing modalities, including electrochemical tests, photochemical tests, electrochemiluminescent tests, and/or any other suitable testing modality.

FIG. 2C illustrates a side view of the test strip of FIG. 2A, according to an exemplary disclosed embodiment. As shown, the test strip 16 has a first test strip substrate 24 and a second test strip substrate 26 that form the bottom or the top portions of the test strip 16. In some embodiments, the test strip substrates 24, 26 can be formed separately and then mated to one another. The substrates 24, 26 can be attached to one another using any suitable means, such as, for example, glue, epoxy, or mechanical attachments (e.g., pins, clips, etc). Further, in some embodiments the test strip substrates 24, 26 may be welded, burned, or attached to one another using any other suitable thermal, mechanical, or chemical bonding methods. In some embodiments, the test strip substrates 24, 26 may be mated to each other such that cavity 17 is substantially hermetically sealed between them.

The test strip substrates 24, 26 can be produced from a variety of suitable material types. For example, in some embodiments, the test strip substrates 24, 26 can include suitable plastics, metals, ceramic, and/or composite materials. Substrates 24, 26 may be selected based on a variety of factors, including for example, cost, processing, feasibility of sterilization, mechanical properties, and effects on enzymes, mediators, or other chemicals needed to produce a suitable reaction. Further, suitable substrates can include a single layer material or multi-layered material.

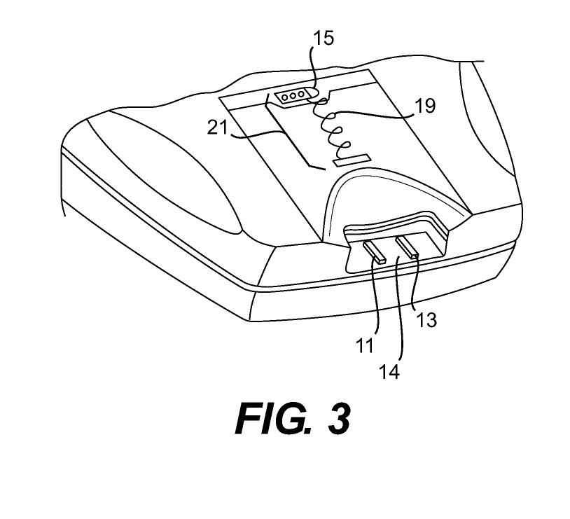

In some embodiments, the analyte test meter 10 of the present disclosure can include an actuation mechanism 21 configured to advance the lancet 18 through the opening 20 in the test strip 16 in order to pierce a patient's skin. FIG. 3 illustrates a perspective view of an exemplary embodiment of a test meter 10 with an actuation mechanism 21. As shown, the actuation mechanism 21 can include a spring 19 configured to quickly push the lancet 18 in and out of the opening 20. The spring 19 can include a variety of suitable spring types, including buckling columns, nested compression springs, conical springs, variable-pitch springs, snap-rings, double torsion springs, wire forms, limited-travel extension springs, braided-wire springs, etc. Further, the spring can be produced from any of a number of metals, plastics, or composite materials.

Further, although as shown, the actuation mechanism 21 is a spring-driven actuation mechanism, any suitable actuation mechanism can be used. For example, as shown, actuation mechanism 21 may alternatively or additionally include a motor 15, such as an electric motor that may control movement of the spring 19 or directly engage the lancet 18. Further, other suitable actuation mechanisms can include a solenoid or other linear actuator, which may advance and retract suitable materials having certain magnetic properties.

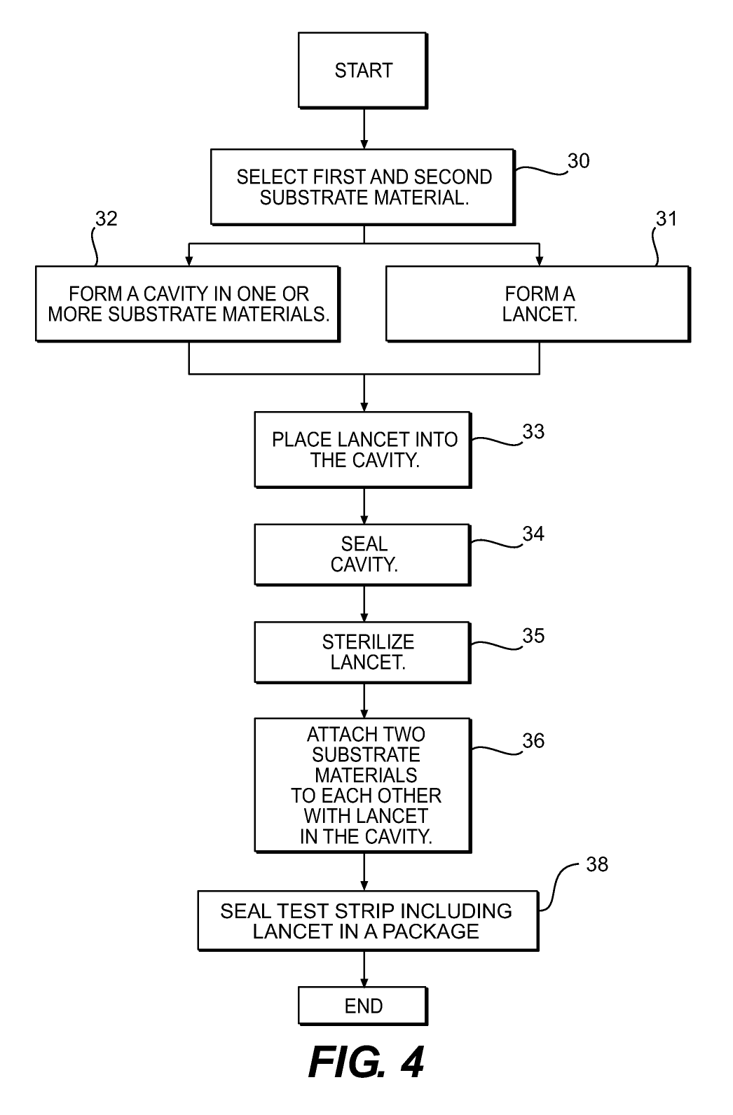

The disclosed test strip 16 with the integrated lancet 18 can be produced using a number of suitable fabrication processes. For example, FIG. 4 illustrates a flow chart of a method of making a test strip with an integrated lancet 18, according to an exemplary disclosed embodiment. As shown at Step 30, first and second substrate materials 24, 26 are first selected. As noted above, these substrate materials can be selected from a variety of different plastics, polymers, metals, ceramics or composite materials. Next, as shown at Step 31, a lancet 18 may be produced with a sharp distal end 41 and a proximal end 42 configured to engage a meter actuation system.

Next, as shown at Step 32, a cavity 17 may be formed in one or more substrates 24, 26. The cavity 17 can be formed using a number of suitable production processes. For example, the cavity 17 can be etched using a laser or water-jet cutter. The cavity 17 can also be formed using a chemical that chemically reacts with at least one of the test strip substrates 24, 26 to remove a selected portion of the substrate, thereby forming a cavity in the substrate. In addition, the cavity 17 can also be formed by using a saw or another cutting tool to cut out a cavity from at least one of the test strip substrates 24, 26. Any suitable method may be selected as long as the cavity has a size and shape configured to hold the lancet 17 and allow the lancet to be moved in and out of the opening 20 to pierce a patient's skin.

Next, as shown at Step 33, the lancet 18 is placed into the cavity 17. As shown at Step 36, the first and second substrates 24, 26 may then be attached to one another, as described above, forming a test strip 16. As noted, test strip substrates 24, 26 may be mated to each other such that cavity 17 is substantially hermetically sealed between them. Further, in some embodiments, the cavity 17 may be hermetically sealed, for instance with a membrane 37, as shown at Step 34.

Additionally, the lancet 18 and/or substrates 24, 26 can be sterilized. The lancet 18 may be sterilized before the test strip 16 is completed, or alternatively, the lancet 18 may be sterilized before sealing the opening 20 or before placement within the cavity 17, as long as the final product will be suitably sanitary for patient use. The lancet may be sterilized after being disposed in first or second substrate material along with the one first or second substrate. Further, in some embodiments, the entire test strip 16, including both test strip substrates 24, 26 may be sterilized in one step after the first and second substrates 24, 26 are attached to one another. In this case, the sterilization process may be performed before application of selected enzymes or mediators, or using a process that will not damage the enzymes or mediators. The lancet may be sterilized using a number of different sterilization techniques including, for example, autoclaving, radiation, ethylene oxide, and ethyl alcohol.

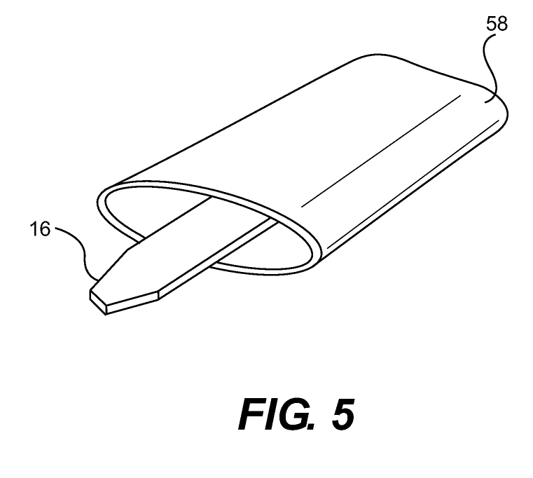

After sterilization, the test strip 16 including the integrated lancet 18 may be sealed in a package 58, as shown at Step 38. Package 58 may be any suitable container or enclosure for test strip 16, such as, for example, a pouch (as depicted in FIG. 5), a box, a case, a sleeve, a packet, or a bag. Package 58 may also comprise one or more thin covers or films. For example, package 58 may comprise one or more covers or wraps disposed on one or more surfaces of test strip 16 and configured to be peeled off test strip 16 at the time of use. Portions of package 58 may be heat-sealed, pressure-sealed, adhesive-sealed, or sealed by other means known in the art. Further, package 58 may be made of a plastic and/or a foil material, such as, for example, a metal foil.

In some embodiments, package 58 containing test strip 16 may be hermetically sealed to prevent entry of fluids and gases. Accordingly, in some embodiments, test strip 16 may be made and/or sealed in package 58 within a sterile and/or controlled environment, such as, for example a dry room. Package 58 may maintain sterility of test strip 16, preventing exposure of test strip 16 to contaminants (e.g., fungi, bacteria, viruses, spore forms, etc.). Additionally, in some embodiments, package 58 may act as a moisture barrier, isolating test strip 16 from ambient humidity. Exposure to water vapor could cause damage to or alter the properties of one or more of the substances or chemicals disposed in test site 22 that are configured to react with one or more analytes. To further limit or eliminate moisture, in some embodiments, package 58 may include a desiccant 60. Suitable desiccants may include silica gel, calcium sulfate, calcium chloride, montmorillonite clay, and molecular sieves. Furthermore, in some embodiments, desiccant 60 may be disposed on a surface within package 58 prior to placement of test strip 16 within package 58. In some embodiments, desiccant 60 may be positioned on a surface within package 58 such that desiccant 60 will be adjacent to test site 22 when test strip 16 is sealed in package 58. This may enhance the effectiveness of desiccant 60 for limiting the moisture exposure of the chemicals disposed in test site 22.

In some embodiments, one test strip 16, including the integrated lancet 18, may be individually sealed into package 58. In this manner, test strip 16 may remain sealed in package 58 until a person opens package 58 in order to use test strip 16 for analyte measurement. This may help ensure both the sterility of test strip 16 and integrity of the chemicals disposed in test site 22 until test strip 16 is actually used. In other embodiments, more than one test strip 16 may be sealed into a single package 58.

The test strip of the present disclosure is straightforward to use. First, a person will engage the test strip 16 with the meter interface 14 to secure the strip in place and form a connection between the electrical components and the test strip lancet and meter actuation system. Next, the user will place his or her skin proximate the test strip opening 20 and actuate the lancet using the meter to pierce the skin and obtain a sample of blood. The blood will be collected on the sample reaction site 22, and the analyte meter 10 will then analyze the blood for analyte concentration and display the results on the display unit 12.

A feature of the present test strip with integrated lancet is the consolidation of the testing components into a single disposable test strip device 16. The consolidated test strip and meter with actuation system provide a more convenient and user friendly system with fewer components. Having a lancet 18 incorporated into a test strip 16 ensures that a patient will use a single lancet 18 only once before disposing it. This method allows the patient to use a sterile lancet 18 every time he or she needs to obtain a sample of blood. Using a lancet 18 only once will also lessen the pain the patient might feel as he or she will use a new, sharp lancet each time rather than re-using the same lancet that might have become dull over time.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### LIST OF ELEMENTS

### Title: Test Strip with Integrated Lancet

10 analyte meter
11 electrode
12 display system
13 electrode
14 test strip interface
15 motor
16 test strip
17 cavity
18 lancet
19 spring
20 opening for lancet's sharp end
21 actuation mechanism
22 reaction test site
24 first test strip substrate
26 second test strip substrate
27 test strip electrodes
30 step of selecting first and second substrate
31 step of forming a lancet
32 step of forming a cavity in one or more substrate
33 step of placing lancet into the cavity
34 step of sealing the cavity
35 step of sterilizing the lancet
36 step of attaching two substrates to each other with lancet in the cavity
37 thin film covering opening of lancet
38 step of sealing the test strip with integrated lancet in a package
40 opening for lancet's mating end
41 sharp end (distal end of lancet)
42 lancet's mating end (proximal end of lancet)
43 second end of test strip
54 first end of test strip
58 package
59 desiccant

1. An analyte test strip system comprising:
an analyte test strip including:
a first test strip substrate,
a second test strip substrate,
an elongate cavity extending through at least one of the first substrate and the second substrate,
a lancet disposed in the at least one elongate cavity,
an opening disposed at one end of the test strip and communicating with the at least one cavity, and
a reaction test site; and
a sealed package containing the analyte test strip.

2. The analyte test strip system of claim 1, wherein the sealed package comprises at least one peelable cover.

3. The analyte test strip system of claim 1, wherein the sealed package comprises a pouch.

4. The analyte test strip system of claim 3, wherein the pouch comprises plastic.

5. The analyte test strip system of claim 4, wherein the pouch comprises foil.

6. The analyte test strip system of claim 1, further comprising a desiccant.

7. The analyte test strip system of claim 6, wherein the desiccant is silica gel.

8. The analyte test strip system of claim 6, wherein the desiccant is a molecular sieve.

9. The analyte test strip system of claim 6, wherein the desiccant is disposed on a surface of the sealed package.

10. The analyte test strip system of claim 9, wherein the desiccant is disposed adjacent to the reaction test site.

11. The analyte test strip system of claim 1, wherein the sealed package contains a plurality of test strips.

12. A method of producing an analyte test strip system, comprising:
selecting a first test strip substrate;
selecting a second test strip substrate;
forming an elongate cavity in at least one of the first test strip substrate or second test strip substrate;
selecting a lancet having at least one sharp end;
positioning the lancet in the at least one elongate cavity;
attaching the first and second substrates to one another forming a test strip, which includes the lancet;
sterilizing the lancet; and
sealing the test strip including the lancet in a package.

13. The method of claim 12, further including sterilizing the lancet before positioning the lancet in the at least one elongate cavity.

14. The method of claim 12, further including sterilizing the test strip and lancet after the lancet is disposed in the cavity of the test strip.

15. The method of claim 12, wherein the sealed package comprises at least one peelable cover.

16. The method of claim 12, wherein the sealed package comprises a pouch.

17. The method of claim 12, wherein the pouch comprises plastic.

18. The method of claim 12, wherein the pouch comprises foil.

19. The method of claim 12, further comprising sealing a desiccant in the package.

20. The method of claim 19, wherein the desiccant is silica gel.

21. The method of claim 19, wherein the desiccant is a molecular sieve.

22. The method of claim 19, further comprising disposing the desiccant on a surface of the sealed package.

23. The method of claim 22, further comprising disposing the desiccant in a position configured to be adjacent to the reaction test site.

24. The method of claim 12, wherein the step of sealing the test strip including the lancet in a package further comprises sealing a plurality of test strips in the package.
